(19) United States
(12) Patent Application Publication
(10) Pub. No.: US 2015/0124215 A1
(43) Pub. Date: May 7, 2015

(54) EYE TRACKING APPARATUS AND IMAGE CAPTURE MODULE THEREOF

(71) Applicant: PIXART IMAGING INC., HSIN-CHU (TW)

(72) Inventors: HUI-HSUAN CHEN, HSIN-CHU (TW); JIA-HONG HUANG, HSIN-CHU (TW); WEN-YEN SU, HSIN-CHU (TW)

(21) Appl. No.: 14/309,296

(22) Filed: Jun. 19, 2014

(30) Foreign Application Priority Data
Nov. 6, 2013 (TW) 102220709

(51) Int. Cl. A61B 3/113

(52) U.S. Cl. CPC A61B 3/113
USPC 351/206

(57) ABSTRACT

An eye tracking apparatus and an image capture module thereof for capturing an iris image are provided. The image capture module includes a carrier, a light source and an image sensing unit. The light source and the image sensing unit are both disposed on the carrier and fastened ahead of the eye by carrier. The light source emits a light beam to the eye, and the image sensing unit receives the light beam reflected from the eye to capture the iris image. The light source and the image sensing unit are arranged at two different sides of a longitudinal reference plane.

### BACKGROUND OF THE INVENTION

1. Field of the Invention

The present invention relates to an eye tracking apparatus and the device thereof; in particular, to an eye tracking apparatus and an image capture module implemented to the eye tracking apparatus.

2. Description of Related Art

The eye-tracking technology is used to track the movement of an eyeball and to detect the point of gaze of a user. The eye-tracking technology is used in medical equipment, an eye tracker, an eye mouse and a head mounted display (HMD).

The conventional eye tracking apparatus usually includes light sources and a camera. Each light source is used to emit a light beam to impinge upon one of the eyes. The camera receives the light beam reflected from the eye and captures the pupil and iris images. The iris image could be obtained by using the image processing to recognize the position of the iris and to measure the movement of the eyeball.

However, the iris colors of humans may not be the same. If one user has lighted-colored iris, the eye tracking apparatus would capture the iris image having the blurrier outer edge because the grayscale of the outer edge of the iris image may be similar to that of the sclera image during the image processing. Accordingly, the movement of the eyeball is not easily determined by the eye tracking apparatus.

### SUMMARY OF THE INVENTION

The instant disclosure provides an eye tracking apparatus, in which the positions of both a light source and an image sensing unit are specially arranged to obtain the sharper iris image.

The instant disclosure provides an image capture module, which is implemented in the abovementioned eye tracking apparatus.

According to an embodiment of the instant disclosure, an image capture module is provided to capture an iris image form an eye. The image capture module includes a carrier, a light source and an image sensing unit. The light source and the image sensing unit are disposed on the carrier and arranged in front of an eye. The light source is used to emit a light beam to an eye, and the image sensing unit receives a reflected light beam from the eye and captures an iris image. The image sensing unit and the light source are arranged different sides of a longitudinal reference plane passing through the center of the eye.

According to another embodiment of the instant disclosure, an eye tracking apparatus for sensing the movement of an iris of an eye is provided. The eye tracking apparatus includes the abovementioned image capture module and a processing unit. The processing unit receives and processes the eye image data from the image sensing unit of the image capture module to obtain the iris image.

In order to further the understanding regarding the instant disclosure, the following embodiments are provided along with illustrations to facilitate the disclosure of the instant disclosure.

### BRIEF DESCRIPTION OF THE DRAWINGS

FIG. 1 shows a front view of an eye tracking apparatus in accordance with one embodiment of the instant disclosure;

FIG. 2A shows a schematic view of an eye tracking apparatus disposed in front of an eye according to an embodiment of the instant disclosure;

FIG. 2B shows a sectional view taken along a line A-A in FIG. 2A;

FIG. 3 shows the grayscale curves of iris images, which are captured by the eye tracking apparatus in accordance with one embodiment of the instant disclosure and by a conventional eye tracking apparatus respectively, taken along the line A-A;

FIG. 4A and FIG. 4B show partial schematic views of eye tracking apparatuses in accordance with embodiments of the instant disclosure;

FIG. 5A and FIG. 5B show partial schematic views of eye tracking apparatuses in accordance with embodiments of the instant disclosure.

### DETAILED DESCRIPTION OF THE EMBODIMENTS

The aforementioned illustrations and following detailed descriptions are exemplary for the purpose of further explaining the scope of the present invention. Other objectives and advantages related to the present invention will be illustrated in the subsequent descriptions and appended drawings.

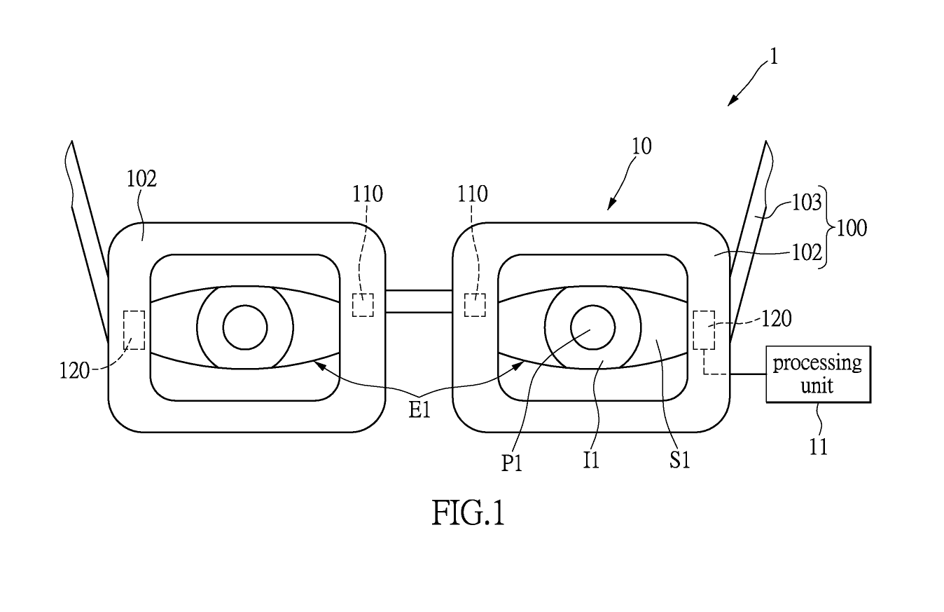

FIG. 1 shows a front view of an eye tracking apparatus in accordance with one embodiment of the instant disclosure. The eye tracking apparatus 1 includes an image capture module 10 and a processing unit 11. The image capture module 10 includes a carrier 100, a light source 110 and an image sensing unit 120. The light source 110 and the image sensing unit 120 are disposed on the carrier 100 by adhering, screwing or mechanical fastening.

Specifically, the eye tracking apparatus 1 may be applied for an electronic device such as a glasses type display or a head mounted display and the like. While a user is using the aforementioned electronic device, the carrier 100 is used to make the light source 110 and an image sensing unit 120 arrange in front of the user's eye E1. In one embodiment of the instant disclosure, the carrier 100 may be an eyeglasses frame which includes at least a frame 102 and a fixing portion. For example, the fixing portion includes a pair of temples 103. User can wear the carrier 100 by utilizing the temples 103 so as to arrange the light source 110 and the image sensing unit 120 ahead of the user's face.

In addition, in the embodiment of FIG. 1, the carrier 100 includes the pair of frames 102. However, in other embodiment, the carrier 100 can include only one frame 102. Each of the temples 103 is connected to the frame 102. For instance, the temple 103 can be pivotally connected to the frame 102 so that the temple 103 can rotate relative to the frame 102. In another case, the temples 103 can be fastened on the frames 102, thus the temples 103 cannot rotate relative to the frame 102. Furthermore, the frames 102 and temples 103 can be integrated to one.

The description of the above mentioned carrier 100 is just for the illustration, and not for limiting the scope of the instant disclosure. For example, in another embodiment, the eye tracking apparatus 1 of the instant disclosure may be applied for the head mounted display. In such circumstance, the fixing portion of the carrier 100 may be a supporting bar so that the frame 102 is hung ahead of the user's eyes.

The processing unit 11 may be a digital signal processor (DSP) which is used to process the image captured by the image sensing unit 120, and to obtain the movement direction of the eye E1. According to the analysis of the movement of the eye E1, the processing unit 11 could recognize the moving direction of the user's visual line. For example, after receiving the image through the image sensing unit 120, the processing unit 11 may analyze the received image by using contour matching to determine the relative distance between the outer edge of the iris and the orbit, and to investigate the moving direction of the user's visual line. The case of the eye tracking apparatus 1 located in front of one eye is described as follows.

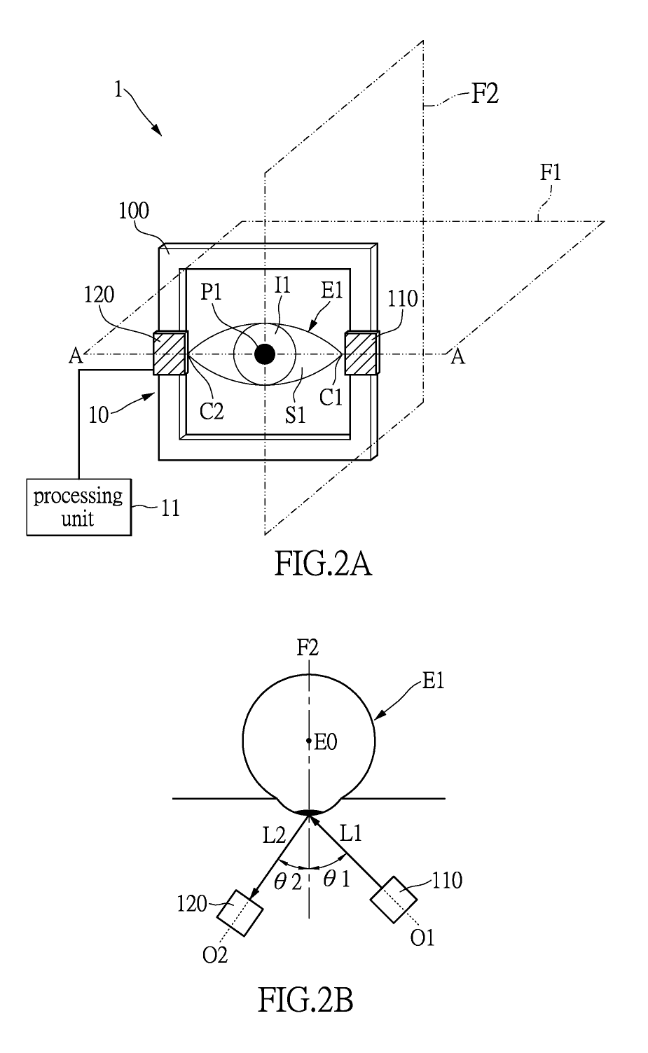

Please refer to FIGS. 2A and 2B. FIG. 2A illustrates the schematic view of the eye tracking apparatus disposed in front of an eye according to an embodiment of the instant disclosure. FIG. 2B shows a sectional view taken along a line A-A in FIG. 2A. The eye E1 has a transverse plane F1 and a longitudinal reference plane F2 perpendicular to the transverse plane F1. In the instant embodiment of the instant disclosure, both of the transverse plane F1 and the longitudinal reference plane F2 pass through a center of the eye E1, and the longitudinal reference plane F2 is located between an inner corner C1 and an outer corner C2 of the eye E1.

Specifically, the transverse plane F1 and the longitudinal reference plane F2 intersect at a center line. The center line passes through the center of the eye E1 which is defined as a center point of pupil P1 when the user looks straight ahead. Furthermore, in the instant embodiment, the line A-A in the transverse plane F1 passes through the center of the eye E1 and is parallel to the normal line of the longitudinal reference plane F2. Moreover, referring to FIG. 2B, both of the transverse plane F1 and the longitudinal reference plane F2 pass through an eyeball center E0 of the eye E1, i.e., the center line passes through the eyeball center E0 as well as the center of the eye E1.

The light source 110 is used to emit a light beam L1 to the eye E1. In one embodiment, the light source 110 may be a light emitting diode (LED). The light beam L1 emitted by the light source 110 may be visible light or invisible light, in which the invisible light such as infrared light. Specifically, the light source 110 may be an infrared LED.

In the instant embodiment, as shown in FIGS. 2A and 2B, the eye tracking apparatus 1 may emit only one light beam L1 to one eye E1. However, in another embodiment, the eye tracking apparatus 1 may emit a plurality of the light beams to one eye E1 so as to generate several glints (not shown in FIG. 2A) on the eye E1, and some of the glints may be located on the outside of the pupil P1. For that reason, the eye tracking apparatus 1 may include a plurality of light sources 110 for emitting the light beams to the eye E1.

The image sensing unit 120 captures the image of the iris I1 by receiving the light beam L2 reflected from the eye E1. Accordingly, the image sensing unit 120 can capture the light beam having the same wavelength as the light beam L1 to form the image. That is, the image sensing unit 120 may be visible light image sensor or IR image sensor. In addition, in another embodiment, the image sensing unit 120 may be a complementary metal-oxide-semiconductor sensor (CMOS Sensor) or a charge-coupled device (CCD).

Please refer to FIG. 2B. In an embodiment of the instant disclosure, concerning one eye E1, the image sensing unit 120 and the light source 110 are respectively arranged in different sides of the longitudinal reference plane F2. In other words, the light source 110 and the image sensing unit 120 may be respectively arranged at the side near to the inner corner C1 and the side near to outer corner C2 of the eye E1.

Specifically, the light source 110 has a first optical axis O1, and the image sensing unit 120 has a second optical axis O2. The first optical axis O1 and the longitudinal reference plane F2 form a first angle θ1. The second optical axis O2 and the longitudinal reference plane F2 form a second angle θ2. The first angle θ1 and the second angle θ2 are acute angle, and the first angle θ1 is larger than or equal to the second angle θ2. That is to say, the first angle θ1 and the second angle O2 satisfy the following mathematic relationship: 90°>θ1≧θ2.

Specifically, the first optical axis O1 forms a first vector projection on the longitudinal reference plane F2. The first angle θ1 is an included angle formed between the first optical axis O1 and the first vector projection. Similarly, the second optical axis O2 forms a second vector projection on the longitudinal reference plane F2, and the second angle θ2 is an included angle formed between the second optical axis O2 and the second vector projection.

In one embodiment, the first angle θ1 and the second angle θ2 are both acute angles and satisfy the mathematic relationship: θ1=θ2±20°, i.e., the first angle θ1 may be smaller than the second angle θ2. In addition, in another embodiment, the first angle θ1 may satisfy the relationship: 0°<θ21≦70°, and the second angle θ2 may satisfy the relationship: 0°<θ2≦70°. The aforementioned arrangement of the light source 110 and the image sensing unit 120 may prevent the light beam L1 or the reflected light beam L2 from being shielded. Accordingly, it is easy for the light beam L1 generated by the light source 110 to project on the eye E1 and for the reflected light beam L2 to be received by the image sensing unit 120.

Based on the above mention, the light beam L1 emitted by the first light source 110 may project on the eye E1 from one side of the longitudinal reference plane F2, and be reflected from the eye E1 to form the reflected light beam L2. The reflected light beam L2 is emitted out from the other side of the longitudinal reference plane F2. While the image sensing unit 120 and the light source 110 are respectively arranged at the different sides of the longitudinal reference plane F2, the illumination of the reflected light beam L2 received by the image sensing unit 120 may be higher so that the image sensing unit 120 can capture the image with higher contrast.

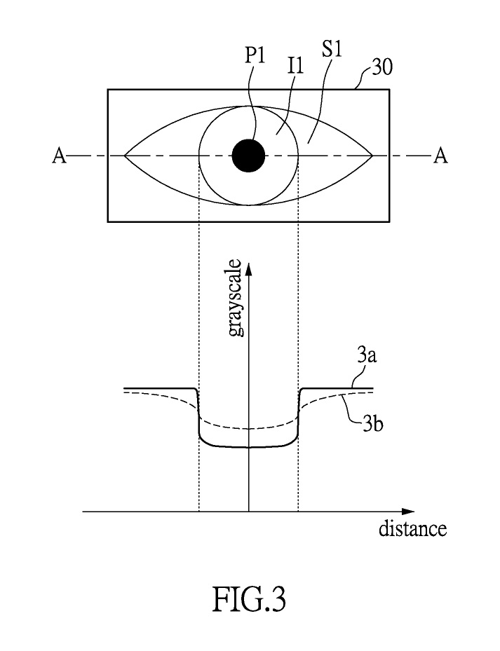

Please refer to FIG. 3. FIG. 3 shows the grayscale variation of the iris images captured by the eye tracking apparatus in accordance with one embodiment of the instant disclosure and captured by a conventional eye tracking apparatus respectively. The curves 3a and 3b shown in FIG. 3 represent the variations of the grayscale value taken along the line A-A shown in the iris image.

The curves 3a and 3b are obtained by measuring the same user's eye. The user is Caucasian whose eye in test is in lighted-colored iris. The curve 3a illustrates the variation of the grayscale value taken along the line A-A shown in the iris image, which is captured by the eye tracking apparatus 1 of the instant disclosure. Compared to curve 3b, it is obvious that the curve 3a illustrates a sharper step between the iris I1 region and the sclera S1 region so that the image captured by the image sensing unit 120 shows the iris I1 with a sharper outer edge. That is to say, when the processing unit 11 receives the data from the image sensing unit 120 and performs image process, it is not necessary for the processing unit 11 to perform the complex processes, such as adjusting the contrast or filtering out noise, for determining where the outer edge of the iris I1 is.

The curve 3b illustrates the variation of the grayscale value taken along the line A-A shown in the iris image, which is captured by the conventional eye tracking apparatus. The curve 3b illustrates the iris image, which is captured by the conventional image sensing unit, with blurry outer edge. It is thus the more complex image processes needs to be performed by the conventional processing unit for the image captured by the conventional image sensing unit so as to find the boundary between the iris I1 and the sclera S1. Accordingly, compared to the conventional eye tracking apparatus in which the light source and the image sensing unit are arranged at the same side of the longitudinal reference plane F2, when the user has lighted-colored iris I1, and the light source 110 and the image sensing unit 120 are arranged at different sides of the longitudinal reference plane F2 in the eye tracking apparatus 1 of the instant disclosure, the image sensing unit 120 may obtain the image, which shows higher contrast between the iris I1 and the sclera S1 and shows the sharper iris image.

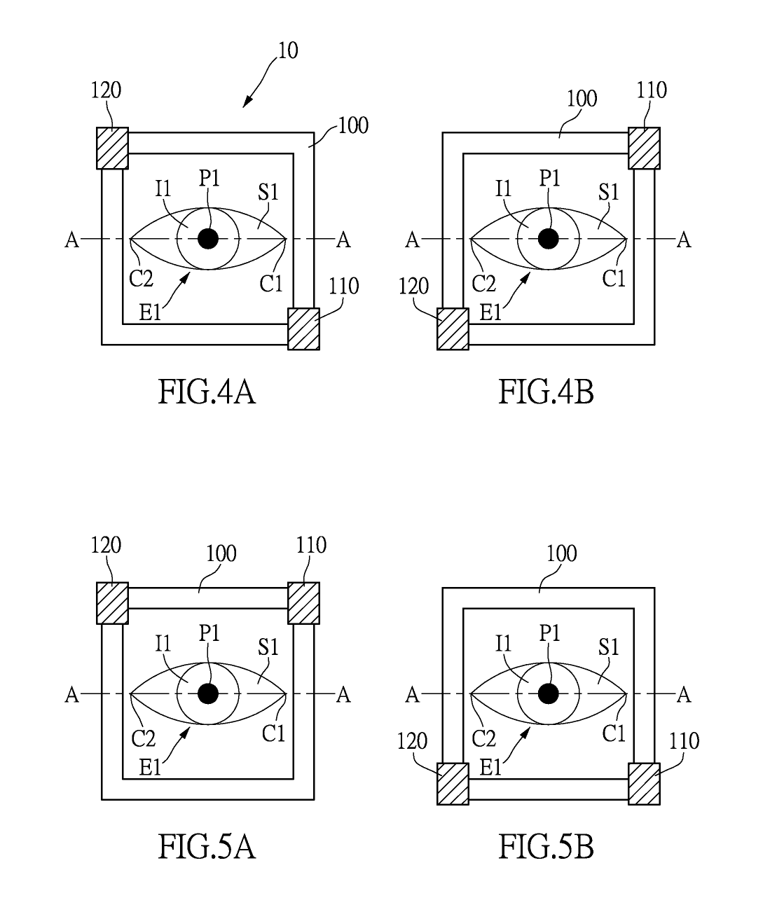

Please refer to FIGS. 4A and 4B. FIGS. 4A and 4B show a partial schematic view of an eye tracking apparatus in accordance with one embodiment of the instant disclosure. In the embodiment of the instant disclosure, when the light source 110 and the image sensing unit 120 are disposed on the carrier 100 ahead of the eye E1, the light source 110 and the image sensing unit 120 are located on the different sides of the transverse plane F1. For example, the light source 110 may be located below the transverse plane F1, and the image sensing unit 120 may be located above the transverse plane F1, as shown in FIG. 4A.

Specifically, the light source 110 is disposed below an imaginary line extending from the inner corner C1 to the outer corner C2, while the image sensing unit 120 is disposed above the imaginary line. In addition, the light source 110 is located near to the inner corner C1, and the image sensing unit 120 is located near to the outer corner C2. In other words, when the user wears the electronic device implementing the eye tracking apparatus 1 of the instant disclosure, the light source 110 is located near to the nose bridge, and the image sensing unit 120 is located far away from the nose bridge. Furthermore, in another embodiment, the locations of the light source 110 and the image sensing unit 120 may be changed to each other. That is to say, the light source 110 may be arranged above the transverse plane F1 and near to the outer corner C2, whereas the image sensing unit 120 may be arranged below the transverse plane F2 and near to the inner corner C1.

Please refer to FIG. 4B. In the present embodiment, the light source 110 is arranged above the transverse plane F1, and the image sensing unit 120 is arranged below the transverse plane F1. Specifically, the light source 110 is disposed at the upper side of the imaginary line extending between the inner corner C1 and the outer corner C2, whereas the image sensing unit 120 is disposed at the lower side of the imaginary line. Similar to the embodiment shown in FIG. 4A, the light source 110 is arranged near to the inner corner C1, and the image sensing unit 120 is arranged near to the outer corner C2.

Please refer to FIGS. 5A and 5B. FIGS. 5A and 5B illustrate partial schematic views of the eye tracking apparatus in accordance with one embodiment of the instant disclosure. In the embodiment of the instant disclosure, the light source 110 and the image sensing unit 120 are arranged at the same side of the transverse plane F1. For example, the light source 110 and the image sensing unit 120 are both located above the transverse plane F1, i.e., at the upper side of the imaginary line extending from the inner corner C1 to the outer corner C2, as shown in FIG. 5A. Moreover, both of the light source 110 and the image sensing unit 120 may be located below the transverse plane F1, i.e., at the lower side of the imaginary line, as shown in the FIG. 5B.

In summary, in the embodiment of the instant disclosure, the light source and the image sensing unit are arranged at different sides of the longitudinal reference plane respectively. It thus causes that the eye tracking apparatus may obtain sharper iris image compared to the conventional eye tracking apparatus. Even for capturing the eye image from the eye having lighted-colored iris, the eye tracking apparatus may obtain a sharper and clearer iris image to analyze. When the processing unit performs the image process for the eye images captured by the image sensing unit to determine the moving direction of the sight line of the user, the processes performed by the processing unit may be simplified, and mistakes due to the lower contrast between the iris and the sclera may be attenuated so that the accuracy for detecting the eye movement may be improved.

The descriptions illustrated supra set forth simply the embodiments of the present invention; however, the characteristics of the present invention are by no means restricted thereto. All changes, alternations, or modifications conveniently considered by those skilled in the art are deemed to be encompassed within the scope of the present invention delineated by the following claims.

What is claimed is:

1. An image capture module for capturing an iris image of an eye, the image capture module comprising:
a carrier;
a light source disposed on the carrier for emitting a light beam to the eye; and
an image sensing unit disposed on the carrier for capturing the iris image by receiving the light beam reflected from the eye, wherein the light source and the image sensing unit are fastened ahead of the eye by the carrier, and arranged at two different sides of a longitudinal reference plane passing through a center of the eye.

2. The image capture module according to claim 1, wherein the longitudinal reference plane and a first optical axis of the light source form a first angle θ1, and the longitudinal reference plane and a second optical axis of the imaging sensing unit form a second angle θ2, and the first angle θ1 and the second angle θ2 satisfy the mathematic relationship: 90°>θ1≧θ2.

3. The image capture module according to claim 2, wherein the first angle θ1 satisfies the mathematic relationship: 0°<θ1≦70°, and the second angle θ2 satisfies the mathematic relationship: 0°<θ2≦70°.

4. The image capture module according to claim 1, wherein the longitudinal reference plane and a first optical axis of the light source form a first angle θ1; the longitudinal reference plane and a second optical axis of the imaging sensing unit form a second angle θ2; and the first angle θ1 and the second angle θ2 satisfy the mathematic relationship: θ1=θ2±20°.

5. The image capture module according to claim 1, wherein the longitudinal reference plane is perpendicular to a transverse plane of the eye and located between an inner corner and an outer corner of the eye, and the light source and the image sensing unit are both arranged at a same side of the transverse plane.

6. The image capture module according to claim 1, wherein the longitudinal reference plane is perpendicular to a transverse plane of the eye and located between an inner corner and an outer corner of the eye, and the light source and the image sensing unit are respectively arranged at two different sides of the transverse plane.

7. The image capture module according to claim 1, wherein the light beam is invisible light.

8. The image capture module according to claim 1, wherein the light source is an infrared light emitting diode.

9. An eye tracking apparatus for determining an movement of an iris of an eye comprising:
a carrier;
a light source disposed on the carrier for emitting a light beam to the eye;
an image sensing unit disposed on the carrier for capturing the iris image by receiving the light beam reflected from the eye, wherein the light source and the image sensing unit are fastened ahead of the eye by the carrier, and arranged at two different sides of a longitudinal reference plane passing through a center of the eye; and
a processing unit for receiving and processing an eye image data form the image sensing unit to recognize an iris image.

10. The eye tracking apparatus according to claim 9, wherein the longitudinal reference plane and a first optical axis of the light source form a first angle θ1, and the longitudinal reference plane and a second optical axis of the imaging sensing unit form a second angle θ2, and the first angle θ1 and the second angle θ2 satisfy the mathematic relationship: 90°>θ1≧θ2.

11. The eye tracking apparatus according to claim 10, wherein the first angle θ1 satisfies the mathematic relationship: 0°<θ1≦70°, and the second angle θ2 satisfies the mathematic relationship: 0°<θ2≦70°

12. The eye tracking apparatus according to claim 9, wherein the longitudinal reference plane and a first optical axis of the light source form a first angle θ1, and the longitudinal reference plane and a second optical axis of the imaging sensing unit form a second angle θ2, and the first angle θ1 and the second angle θ2 satisfy the mathematic relationship: θ1=θ2±20°.

13. The eye tracking apparatus according to claim 9, wherein the longitudinal reference plane is perpendicular to a transverse plane of the eye and located between an inner corner and an outer corner of the eye.

14. The eye tracking apparatus according to claim 13, wherein the image sensing unit and the light source are arranged above the transverse plane.

15. The eye tracking apparatus according to claim 13, wherein the image sensing unit and the light source are arranged below the transverse plane.

16. The eye tracking apparatus according to claim 13, wherein the image sensing unit and the light source are respectively arranged at two different sides of the transverse plane.

17. The eye tracking apparatus according to claim 9, wherein the light beam is invisible light.

18. The eye tracking apparatus according to claim 9, wherein the light source is an infrared light emitting diode.

19. The eye tracking apparatus according to claim 9, wherein the processing unit is a digital signal processor.

20. The eye tracking apparatus according to claim 9, wherein the carrier is an eyeglasses frame.
